(19) United States
(12) Patent Application Publication
(10) Pub. No.: US 2023/0305025 A1
(43) Pub. Date: Sep. 28, 2023

(54) METHOD OF MEASURING HEMOGLOBIN F

(71) Applicant: ARKRAY, Inc., Kyoto (JP)

(72) Inventor: Kazuki ISHIKAWA, Kyoto-shi (JP)

(73) Assignee: ARKRAY, Inc., Kyoto (JP)

(21) Appl. No.: 18/123,477

(22) Filed: Mar. 20, 2023

(30) Foreign Application Priority Data
Mar. 22, 2022 (JP) 2022-045897

(51) Int. Cl. G01N 33/72; G01N 30/86

(52) U.S. Cl. CPC G01N 33/721; G01N 30/8631; G01N 2030/027

(57) ABSTRACT

A first correlation equation is preliminarily determined from a chromatogram obtained by subjecting, to liquid chromatography, a first blood sample group which is known to contain HbA1c, and whose content ratio of HbF in total hemoglobin is known to be less than a predetermined content ratio, the first correlation equation being a correlation equation between an HbA1c peak value and a composite peak value including an HbA1a peak and an HbA1b peak. A composite peak value obtained by applying, to the first correlation equation, an HbA1c peak value of a chromatogram obtained by subjecting a measurement target blood sample to liquid chromatography is subtracted from a composite peak value including an HbA1a peak and an HbA1b peak in the blood sample, to calculate a modified HbF peak value. The modified HbF peak value is added to an HbF peak value of the blood sample, to correct the HbF peak value.

### CROSS-REFERENCE TO RELATED APPLICATION

This application claims priority under 35 USC 119 from Japanese Patent Application No. 2022-045897, filed on Mar. 22, 2022, the disclosure of which is incorporated by reference herein.

### BACKGROUND

### Technical Field

The present invention relates to a method of measuring hemoglobin F in a blood sample.

### Related Art

Hemoglobin in blood samples can be measured by separation/fractionation methods such as high-performance liquid chromatography. Since a measured value of hemoglobin F (HbF), which is a type of hemoglobin, can be used as a basis for diagnosis of hemoglobinopathy and thalassemia, the value needs to be highly accurate. Examples of a method of measuring hemoglobin F using liquid chromatography include the method disclosed in Japanese Patent Application Laid-Open (JP-A) No. 2014-235023.

A chromatogram obtained by subjecting a blood sample to cation-exchange liquid chromatography shows, in the side with a higher rate of elution from the column relative to the HbF peak, a composite peak formed by overlapping peaks of hemoglobin A1a (HbA1a) and hemoglobin A1b (HbA1b), which are types of hemoglobin A1 (HbA1), which is a glycosylated product of hemoglobin A (HbA).

In view of this, an aspect of the present disclosure provides a technique that enables a more accurate measurement of HbF from a chromatogram obtained by subjecting a blood sample to liquid chromatography.

### SUMMARY

As in the process in which HbA is glycosylated to become HbA1c, it can be assumed that HbF may become modified HbF when it is modified by glycosylation or the like. Since the modified HbF is also a type of HbF, measurement of the peak value of the modified HbF is also required for more accurate measurement of HbF. In an experiment by the present inventors, the size of the composite peak was found to vary in accordance with the size of the HbF peak. From this observation result, it was assumed that the composite peak also includes a modified product of HbF. Since the HbA1a, HbA1b, and modified HbF included in the composite peak are similar to each other in terms of the charge and the size, it is difficult to separate the HbA1a, HbA1b, and modified HbF from each other by cation-exchange chromatography, and hence the peak value of the modified HbF cannot be accurately measured. As a result, it is impossible to measure the total amount of HbF (in other words, the sum of the unmodified HbF concentration and the modified HbF concentration) in the blood sample.

In one mode of the method of measuring HbF of the disclosure, a first correlation equation is preliminarily determined from a chromatogram obtained by subjecting, to liquid chromatography, a first blood sample group which is known to contain HbA1c, and whose content ratio of HbF in total hemoglobin is known to be less than a predetermined content ratio, wherein the first correlation equation is a correlation equation between an HbA1c peak value and a composite peak value including an HbA1a peak and an HbA1b peak. A composite peak value obtained by applying an HbA1c peak value of a measurement target blood sample to the first correlation equation is subtracted from a composite peak value including an HbA1a peak and an HbA1b peak in the blood sample, to calculate a modified HbF peak value. The modified HbF peak value is added to an HbF peak value of the blood sample, to correct the HbF peak value.

According to an aspect of the invention, HbF can be more accurately measured from a chromatogram obtained by subjecting a blood sample to liquid chromatography.

### BRIEF DESCRIPTION OF THE DRAWINGS

Exemplary embodiments will be described in detail based on the following figures, wherein:

FIG. 1 schematically shows an example of a chromatogram of hemoglobin obtained by subjecting a blood sample to high-performance liquid chromatography;

FIG. 2 schematically shows a relationship between an HbF peak and a composite peak in a normal sample;

FIG. 3 schematically shows a relationship between an HbF peak and a composite peak in a high-HbF sample;

FIG. 4 is a chromatogram obtained by subjecting blood sample 1 to high-performance liquid chromatography;

FIG. 5 is a chromatogram obtained by subjecting blood sample 2 to high-performance liquid chromatography;

FIG. 6 is a chromatogram obtained by subjecting blood sample 3 to high-performance liquid chromatography;

FIG. 7 is a chromatogram obtained by subjecting blood sample 4 to high-performance liquid chromatography;

FIG. 8 is a scatter diagram illustrating a correlation between an HbA1c peak value and a composite peak value including an HbA1 a peak and an HbA1b peak in a chromatogram obtained by subjecting a blood sample group of healthy individuals to high-performance liquid chromatography; and

FIG. 9 is a scatter diagram illustrating a correlation between an HbF peak value and a modified HbF peak value in a chromatogram obtained by subjecting a blood sample group that is known to include HbF, to high-performance liquid chromatography.

### DETAILED DESCRIPTION

Embodiments in the disclosure are described below with reference to drawings. The symbols shared among the diagrams represent identical portions even without any description. The “peak value” in the disclosure means the height or area of each peak found in a chromatogram, and the value may be either a relative value or an absolute value. The relative value may be the ratio to the total area of the chromatogram, may be the ratio to the total area of the peaks related to hemoglobin in the chromatogram, or may be the ratio to the area of a specific peak (such as an HbA0 peak).

### (1) First Embodiment

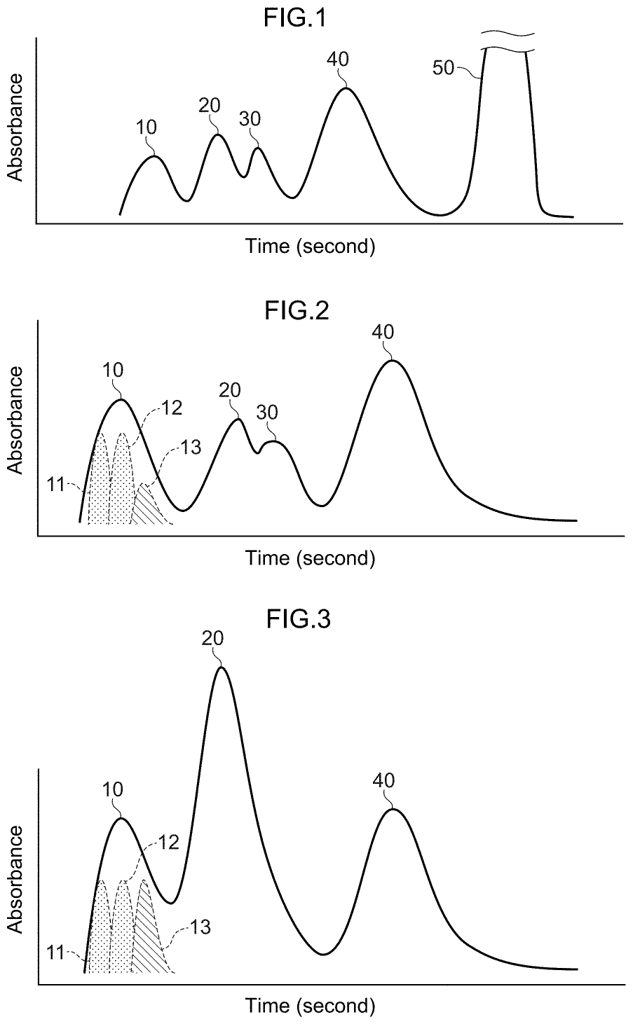

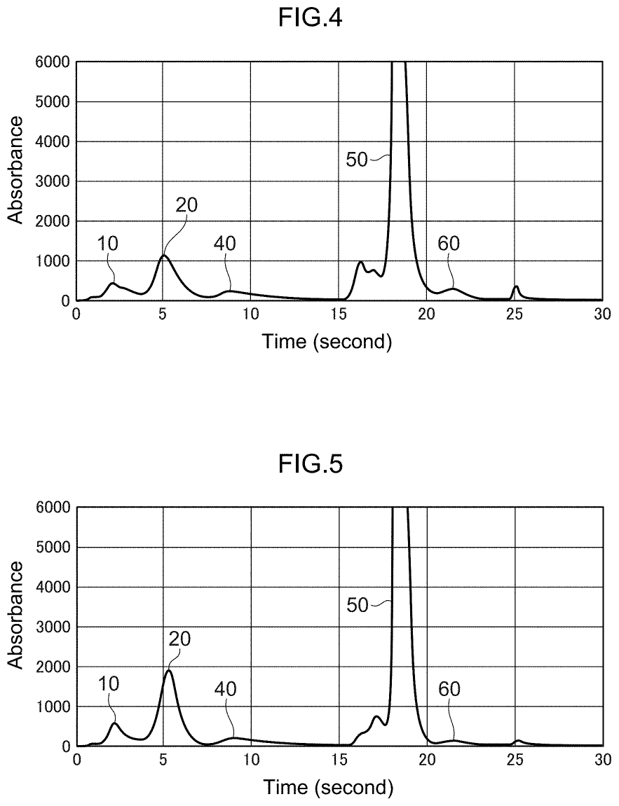

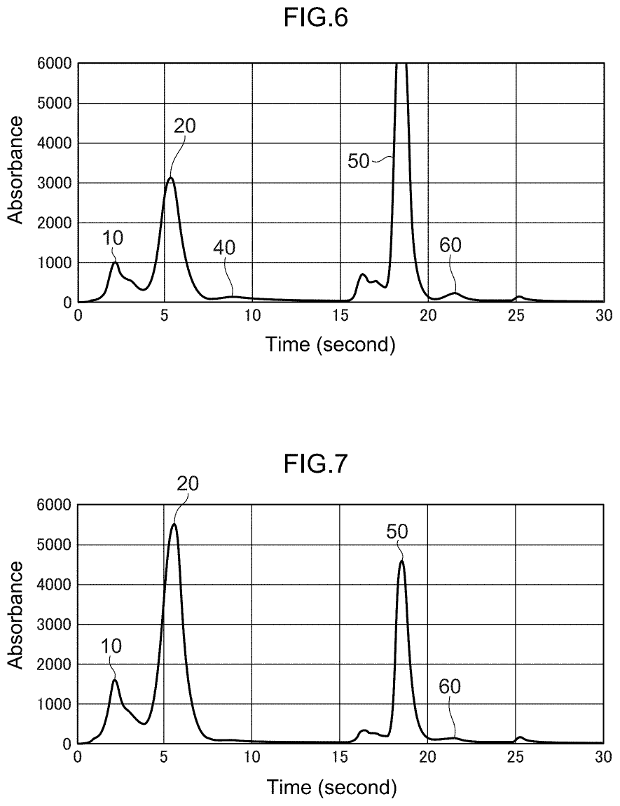

When a blood sample is subjected to cation-exchange liquid chromatography, a chromatogram of hemoglobin such as the one schematically represented in FIG. 1 can be obtained. In this chromatogram, a composite peak 10 including an HbA1a peak 11 and an HbA1b peak 12 (see FIG. 2 and FIG. 3), an HbF peak 20, an unstable HbA1c peak 30, an HbA1c peak 40, an HbA0 peak 50, and an HbA2 peak 60 (see FIG. 4 to FIG. 7) appear in the order of increasing rate of elution from the column.

FIG. 2 schematically shows a chromatogram of a normal blood sample having an HbF peak value of less than 1%, and FIG. 3 schematically shows a chromatogram of a blood sample of a high-HbF patient. As can be seen by comparison between these figures, which show HbA1c peaks 40 of almost the same size, the HbF peak 20 in the chromatogram of FIG. 3 is higher than the HbF peak 20 in the chromatogram of FIG. 2. In this case, the composite peak 10 in the chromatogram of FIG. 3 is also higher than the composite peak 10 in the chromatogram of FIG. 2.

Here, as shown in FIG. 2, the composite peak 10 includes an HbA1a peak 11 and an HbA1b peak 12 of HbA1, which is a glycosylated product of HbA. As shown in FIG. 3, an increase in the HbF peak 20 results in an increase in the composite peak 10. Since the composite peak 10 includes the HbA1a peak 11 and the HbA1b peak 12, which are glycosylated products, the increase in the composite peak 10 is thought to be due to an increase in a modified HbF peak 13, which is a modified product of HbF, as shown in FIG. 3. In contrast, the normal sample shown in FIG. 2 hardly includes the modified HbF peak 13, and most part of the composite peak 10 is thought to be formed by the HbA1a peak 11 and the HbA1b peak 12.

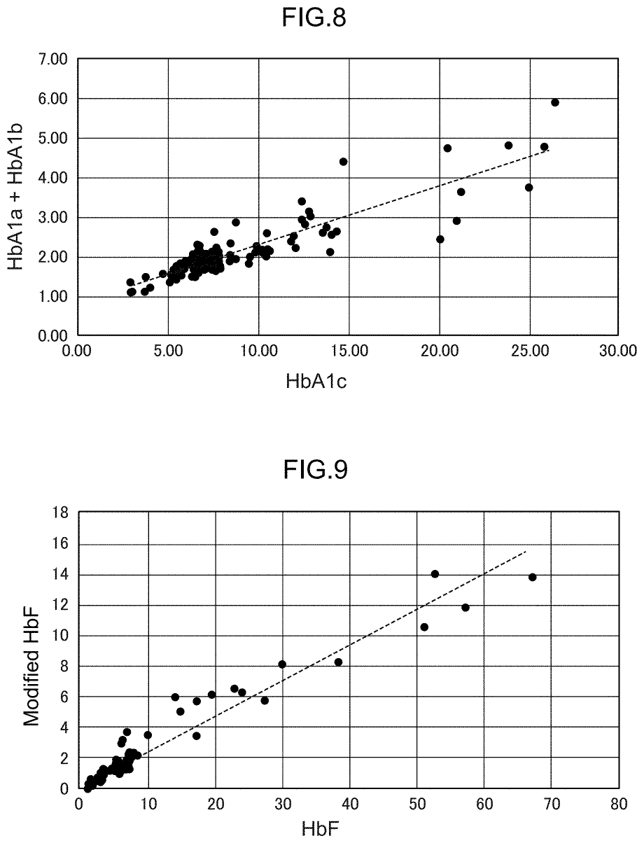

On the other hand, providing that, in both of a normal sample as shown in FIG. 2 and a high-HbF sample as shown in FIG. 3, the ratio of the sum of the HbA1a peak 11 value and the HbA1b peak 12 value to the HbA1c peak 40 is almost constant, if a correlation between the peak value of the HbA1c peak 40 and the peak value of the composite peak 10 (which is substantially the sum of the peak value of the HbA1a peak 11 and the peak value of the HbA1b peak 12) can be discovered, the correlation can be applied to the HbA1c peak 40 of a high-HbF sample to calculate the sum of the HbA1a peak 11 and the HbA1b peak 12 in the composite peak 10. The difference between the sum of the peak value of the HbA1a peak 11 and the peak value of the HbA1b peak 12, and the peak value of the composite peak 10, derives from the peak value of the modified HbF peak 13. It is thought that, by subtracting the calculated sum from the composite peak 10 in the chromatogram shown in FIG. 3, the modified HbF peak 13 can be calculated. As shown in Examples below (see FIG. 8), it was found, for a plurality of normal samples, that there is a high correlation between the HbA1c peak 40 and the composite peak 10.

Based on this correlation, in the method of measuring HbF of a first embodiment of the disclosure, a first correlation equation is preliminarily determined from a chromatogram obtained by subjecting, to liquid chromatography, a first blood sample group which is known to contain HbA1c but not to contain HbF, wherein the first correlation equation is a correlation equation between an HbA1c peak value and a composite peak value including an HbA1a peak and an HbA1b peak. A composite peak value obtained by applying, to the first correlation equation, an HbA1c peak value of a chromatogram obtained by subjecting a measurement target blood sample to liquid chromatography is subtracted from a composite peak value including an HbA1a peak and an HbA1b peak of the blood sample, to calculate a modified HbF peak value. The modified HbF peak value is added to an HbF peak value of the blood sample, to correct the HbF peak value. Since the HbF peak value correlates with the concentration of HbF contained in the blood sample, it is possible to determine the concentration of HbF contained in the blood sample, the ratio of the amount of HbF relative to total hemoglobin, and the like based on the HbF peak value.

More specifically, for a plurality of blood samples which are known to contain HbA1c, and whose content ratios of HbF in total hemoglobin are known to be less than a predetermined content ratio, a first correlation equation is determined from a correlation between the HbA1c peak value and the composite peak value (which is substantially the sum of the HbA1 a peak value and the HbA1b peak value). Note that the “predetermined content ratio” herein may be an HbF peak value of not more than a normal value (for example, 5%, preferably 3%, more preferably 1% relative to the peak value of total hemoglobin). The “plurality of blood samples whose content ratios of HbF in total hemoglobin are known to be less than a predetermined content ratio” may be obtained as blood samples which show HbF peak values of less than the predetermined content ratio as obtained by separation analysis of hemoglobin contained in the blood samples by liquid chromatography. Alternatively, for example, the plurality of blood samples may be obtained as blood samples whose hemoglobin F contents have been shown to be less than a predetermined content ratio by analysis using a measurement principle other than liquid chromatography (for example, capillary electrophoresis). The first correlation equation is as shown in Formula (1) below, wherein the HbA1c peak value is variable x, and the composite peak value is variable y.

y=a₁x+b₁ (1)
x: HbA1c peak value (variable)
y: composite peak value (variable)
a₁: slope (constant)
b₁: intercept (constant)

The HbA1c peak value is then determined from the chromatogram obtained from the measurement target blood sample, and the value is assigned to variable x of Formula (1) above, and the composite peak value is assumed as the calculated variable y. The calculated estimated value of the composite peak is subtracted from the composite peak value obtained from the composite peak 10 that appears in the chromatogram (i.e., the composite peak value measured using the composite peak 10 that appears in the chromatogram), to calculate the value of the modified HbF peak included in the composite peak 10. Then, by adding the calculated modified HbF peak value to the peak value of the HbF peak 20 obtained from the chromatogram, the HbF peak value is corrected to assume the true HbF peak value in the blood sample. The modified HbF peak value is as shown in the following Formula (2) below. When the modified HbF peak value is w, and the HbF peak value is variable z, the true HbF peak value (v) is as shown in the following Formula (3) below.

w=y−(a₁x+b₁) (2)

v=z+w=z+(y−(a₁x+b₁)) (3)
v: true HbF peak value (variable)
w: modified HbF peak value (variable)
x: HbA1c peak value (variable)
y: composite peak value (variable)
z: HbF peak value (variable)
a₁: slope (constant)
b₁: intercept (constant)

### (2) Second Embodiment

The first embodiment shown above is dependent on the presence of HbA1c in the measurement target blood sample, and it is impossible to apply the HbA1c peak value to the first correlation equation in cases where the blood sample does not contain HbA1c, or where HbA1c is not detected in the blood sample.

In view of this, in the method of measuring HbF of a second embodiment, a first correlation equation is preliminarily determined as described in the first embodiment. Further, from a chromatogram obtained by subjecting a second blood sample group which is known to contain HbA1c and HbF to liquid chromatography, the HbA1c peak value is applied to the first correlation equation, to obtain a composite peak value. The composite peak value is subtracted from a composite peak value including an HbA1 a peak and an HbA1b peak in the second blood sample group, to obtain an estimated modified HbF peak value of the second blood sample group. A second correlation equation, which is a correlation equation between an HbF peak value and the estimated modified HbF peak value in the second blood sample group, is preliminarily determined. The HbF peak value of a chromatogram obtained by subjecting the measurement target blood sample to liquid chromatography is applied to the second correlation equation, to calculate the modified HbF peak value. The modified HbF peak value is then added to the HbF peak value of the blood sample, to correct the HbF peak value.

More specifically, first, as described above in the first embodiment, the first correlation equation of Formula (1) shown above is determined.

Subsequently, for chromatograms of a plurality of blood samples which are known to contain both HbA1c and HbF, the HbA1c peak value is determined. The value is then assigned to variable x of Formula (1) shown above. Then, the composite peak value is assumed as the calculated variable y. The calculated composite peak value is subtracted from the composite peak value obtained from the composite peak 10 that appears in the chromatogram (i.e., the composite peak value measured using the composite peak 10 that appears in the chromatogram), to calculate the value of the modified HbF peak included in the composite peak 10. On the other hand, from the same chromatogram, the HbF peak value is obtained, and the second correlation equation is determined from a correlation between the HbF peak value and the calculated modified HbF peak value. The second correlation equation is as shown in Formula (4) below, wherein the HbF peak value is variable z, and the modified HbF peak value is variable w.

w=a₂z+b₂ (4)
w: modified HbF peak value (variable)
z: HbF peak value (variable)
a₂: slope (constant)
b₂: intercept (constant)

The HbF peak value is then determined from a chromatogram obtained from the measurement target blood sample, and the value is assigned to variable z of Formula (4) shown above. Then, the modified HbF peak value is calculated as variable w. By adding the calculated modified HbF peak value to the HbF peak value obtained from the chromatogram, the HbF peak value is corrected to assume the true HbF peak value in the blood sample. The true HbF peak value in the blood sample (v) is as shown in the following Formula (5).

v=z+w=z+(a₂z+b₂) (5)
v: true HbF peak value (variable)
w: modified HbF peak value (variable)
z: HbF peak value (variable)
a₂: slope (constant)
b₂: intercept (constant)

Note that, instead of the second correlation equation shown above, a third correlation equation may be determined from a correlation between the HbF peak value and the corrected HbF peak value (i.e., the sum of the HbF peak value and the modified HbF peak value). The third correlation equation is as shown in the following Formula (6), wherein the HbF peak value is variable z, and the corrected HbF peak value as the true HbF peak value is v.

v=a₃z+b₃ (6)
v: true HbF peak value (variable)
z: HbF peak value (variable)
a₃: slope (constant)
b₃: intercept (constant)

The HbF peak value is then determined from a chromatogram obtained from the measurement target blood sample, and the value is assigned to variable z of Formula (5) or Formula (6) shown above. The corrected HbF peak value, that is, the true HbF peak value in the blood sample, is assumed as the calculated variable v.

### (3) Third Embodiment

The first embodiment is suitable for blood samples containing HbA1c. The second embodiment is suitable for blood samples not containing HbA1c. Therefore, it is desirable to employ the measurement method of the first embodiment or the measurement method of the second embodiment depending on whether HbA1c is included in the chromatogram.

In view of this, in the method of measuring HbF of a third embodiment, the first correlation equation is preliminarily determined as described in the first embodiment, and the second correlation equation is preliminarily determined as described in the second embodiment. Then, in cases where the target chromatogram obtained by subjecting the measurement target blood sample to liquid chromatography has the HbA1c peak, the HbF peak value of the blood sample is corrected as described in the first embodiment. On the other hand, in cases where the target chromatogram obtained by subjecting the measurement target blood sample to liquid chromatography does not have the HbA1c peak, the HbF peak value of the blood sample is corrected as described in the second embodiment.

### Examples

(1) Liquid Chromatography Apparatus

A commercially available cation-exchange chromatography column packed with a hydrophilic polymer containing a methacrylate copolymer was connected to a commercially available high-performance liquid chromatography apparatus. To a predetermined position of the downstream channel of the cation-exchange chromatography column, an optical detector (more specifically, an absorption spectrometer) that detects the concentration of hemoglobin passing through the channel was attached.

(2) Eluent

Three types of eluents were prepared as aqueous solutions having the compositions shown in Table 1 below.

TABLE 1

| | Concentration (mass %) | | |
|---|---|---|---|
| Material | Eluent A | Eluent B | Eluent C |
| Sodium dihydrogen | 1.44 | 0.02 | 0.12 |
| phosphate dihydrate | | | |
| Disodium hydrogen | 0.16 | 0.50 | 0.33 |
| phosphate | | | |

The pH of Eluent A was adjusted to 5.08; the pH of Eluent B was adjusted to 8.0; and the pH of Eluent C was adjusted to 6.82. Regarding the elution strength of these eluents for hemoglobin, Eluent A had the lowest strength, and Eluent B had the highest strength.

(3) Chromatogram

Eluent A was passed through the liquid chromatography apparatus of (1), to equilibrate the column. Thereafter, a hemolyzed blood sample was introduced into the column. Thereafter, Eluent A was passed through the column for 13 seconds, to elute HbA1a, HbA1b, HbF, and HbA1c. Subsequently, a mixed solution of Eluent A and Eluent C at 1:9 was passed through the column for 5 seconds, to elute HbA0. Subsequently, Eluent C was passed through the column for 5 seconds, to elute HbA2. Thereafter, Eluent B was passed through the column for 2 seconds to elute the entire hemoglobin remaining in the column, and then Eluent A was passed through the column for 5 seconds. During the elution, a chromatogram was prepared based on the absorbance obtained by the optical detector at a detection wavelength of 420 nm.

Examples of the obtained chromatogram are shown in FIG. 4 to FIG. 7. As is evident from these figures, as the area of the HbF peak 20 (HbF peak value) increased, the area of the composite peak 10 (composite peak value) increased. Thus, the composite peak 10, which is known to include the HbA1a peak 11 and the HbA1b peak 12 (see FIG. 3), was strongly assumed to include the modified HbF peak value 13 (see FIG. 3).

(4) First Correlation Equation

In order to determine the first correlation equation, blood samples of 66 healthy individuals (blood samples showing HbF peak values of less than 1% relative to the peak values of total hemoglobin) were provided. A chromatogram was obtained from each of these blood samples, and the HbA1c peak value and the composite peak value were measured for the chromatogram. As a result, it was found that there is a correlation as shown in the scatter diagram of FIG. 8. The abscissa of the graph represents the HbA1c peak value, and the ordinate represents the composite peak value (that is, the sum of the HbA1a peak value and the HbA1b peak value). The first correlation equation, represented by the dotted line in FIG. 8, was as shown in Formula (7) below, wherein the HbA1c peak value is variable x, and the composite peak value is variable y. The correlation coefficient (r) of the first correlation equation was 0.8951.

y=0.1475x+0.8048 (7)
x: HbA1c peak value (variable)
y: composite peak value (variable)

It can be assumed that the presence of the correlation between the composite peak 10 including HbA1a and HbA1b, and the HbA1c peak 40, may be due to the fact that the ratios of HbA1a, HbA1b, and HbA1c are almost constant since all of HbA1a, HbA1b, and HbA1c are glycosylated products of HbA0 and are produced as a result of glycosylation of HbA0.

The HbA1c peak value is then measured from the chromatogram obtained from the measurement target blood sample, and the value is assigned as variable x to Formula (7). As variable y, the sum of the HbA1a peak value and the HbA1b peak value (hereinafter referred to as “AB value”) is calculated. The difference between the composite peak value measured from the chromatogram and the AB value derives from the modified HbF peak value. Thus, the AB value is subtracted from the composite peak value measured from the chromatogram, to calculate the modified HbF peak value. Then, the value obtained by adding the calculated modified HbF peak value to the HbF peak value measured from the chromatogram is assumed to be the true HbF peak value of the blood sample.

(2) Second Correlation Equation

In order to determine the second correlation equation, blood samples of 66 healthy individuals (blood samples showing HbF peak values of less than 1% relative to the peak values of total hemoglobin) and blood samples of 48 high-HbF patients were provided. A chromatogram was obtained from each of these blood samples. The HbA1c peak value was measured from the chromatogram, and the value was assigned as variable x to Formula (7), to calculate the AB value as variable y. The AB value was then subtracted from the composite peak value measured from the chromatogram, to calculate the modified HbF peak value. As a result, it was found that, between the HbF peak value measured from the chromatogram and the calculated modified HbF peak value, there is a correlation as shown in the scatter diagram of FIG. 9. The abscissa of the graph represents the HbF peak value, and the ordinate represents the modified HbF peak value. The second correlation equation, represented by the dotted line in FIG. 9, was as shown in Formula (8) below, wherein the HbF peak value is variable z, and the modified HbF peak value is variable w. The correlation coefficient (r) of the second correlation equation was 0.9697.

w=0.2323z (8)

From a chromatogram obtained from the measurement target blood sample, the HbF peak value is measured, and the value is assigned as variable z to Formula (8), to calculate the modified HbF peak value as variable w. The value obtained by adding the calculated modified HbF peak value to the HbF peak value measured from the chromatogram is assumed to be the true HbF peak value of the blood sample.

### INDUSTRIAL APPLICABILITY

The invention is applicable to measurement of HbF in a blood sample using liquid chromatography.

What is claimed is:

1. A method of measuring hemoglobin F, the method comprising:
preliminarily determining a first correlation equation from a chromatogram obtained by subjecting a first blood sample group to liquid chromatography, the first blood sample group being known to contain hemoglobin A1c, and being known to contain less hemoglobin F than a predetermined content ratio relative to total hemoglobin, and the first correlation equation being a correlation equation between a hemoglobin A1c peak value and a composite peak value including a hemoglobin A1a peak and a hemoglobin A1b peak;
calculating a modified hemoglobin F peak value by subtracting a composite peak value obtained by applying, to the first correlation equation, a hemoglobin A1c peak value of a chromatogram obtained by subjecting a measurement target blood sample to liquid chromatography, from a composite peak value including a hemoglobin A1a peak and a hemoglobin A1b peak in the blood sample; and
correcting a hemoglobin F peak value by adding the modified hemoglobin F peak value to the hemoglobin F peak value of the blood sample.

2. A method of measuring hemoglobin F, the method comprising:
preliminarily determining a first correlation equation from a chromatogram obtained by subjecting a first blood sample group to liquid chromatography, the first blood sample group being known to contain hemoglobin A1c, and being known to contain less hemoglobin F than a predetermined content ratio relative to total hemoglobin, and the first correlation equation being a correlation equation between a hemoglobin A1c peak value and a composite peak value including a hemoglobin A1a peak and a hemoglobin A1b peak;
obtaining a composite peak value by applying a hemoglobin A1c peak value to the first correlation equation, the hemoglobin A1c peak value being obtained from a chromatogram obtained by subjecting a second blood sample group known to contain hemoglobin A1c and hemoglobin F to liquid chromatography;
obtaining an estimated modified hemoglobin F peak value of the second blood sample group by subtracting the composite peak value from a composite peak value including a hemoglobin A1a peak and a hemoglobin A1b peak in the second blood sample group;
preliminarily determining a second correlation equation using a hemoglobin F peak value and the estimated modified hemoglobin F peak value of the second blood sample group, the second correlation equation being a correlation equation between a hemoglobin F peak value and a modified hemoglobin F peak value;
calculating a modified hemoglobin F peak value by applying a hemoglobin F peak value of a chromatogram obtained by subjecting a measurement target blood sample to liquid chromatography, to the second correlation equation; and
correcting a hemoglobin F peak value by adding the modified hemoglobin F peak value to the hemoglobin F peak value of the blood sample.

3. A method of measuring hemoglobin F, the method comprising:
preliminarily determining a first correlation equation from a chromatogram obtained by subjecting a first blood sample group to liquid chromatography, the first blood sample group being known to contain hemoglobin A1c, and being known to contain less hemoglobin F than a predetermined content ratio relative to total hemoglobin, and the first correlation equation being a correlation equation between a hemoglobin A1c peak value and a composite peak value including a hemoglobin A1a peak and a hemoglobin A1b peak;
obtaining a composite peak value by applying a hemoglobin A1c peak value to the first correlation equation, the hemoglobin A1c peak value being obtained from a chromatogram obtained by subjecting a second blood sample group known to contain hemoglobin A1c and hemoglobin F to liquid chromatography;
obtaining an estimated modified hemoglobin F peak value of the second blood sample group by subtracting the composite peak value from a composite peak value including a hemoglobin A1a peak and a hemoglobin A1b peak in the second blood sample group; and
preliminarily determining a second correlation equation using a hemoglobin F peak value and the estimated modified hemoglobin F peak value of the second blood sample group, the second correlation equation being a correlation equation between a hemoglobin F peak value and a modified hemoglobin F peak value;
wherein, in a case in which a chromatogram obtained by subjecting a measurement target blood sample to liquid chromatography includes a hemoglobin A1c peak, the method further comprises:
calculating a modified hemoglobin F peak value by subtracting a composite peak value obtained by applying a hemoglobin A1c peak value of the blood sample to the first correlation equation, from a composite peak value including a hemoglobin Ala peak and a hemoglobin A1b peak of the blood sample; and
correcting a hemoglobin F peak value by adding the modified hemoglobin F peak value to the hemoglobin F peak value of the blood sample, or
wherein, in a case in which a chromatogram obtained by subjecting a measurement target blood sample to liquid chromatography does not include a hemoglobin A1 c peak, the method further comprises:
calculating a modified hemoglobin F peak value by applying a hemoglobin F peak value of the blood sample to the second correlation equation; and
correcting a hemoglobin F peak value by adding the modified hemoglobin F peak value to the hemoglobin F peak value of the blood sample.

4. The method of measuring hemoglobin F according to claim 2,
wherein:
a third correlation equation that is a correlation equation between a hemoglobin F peak value and a sum of the hemoglobin F peak value and the estimated modified hemoglobin F peak value in the second blood sample group is preliminarily determined instead of the second correlation equation; and
a hemoglobin F peak value of a chromatogram obtained by subjecting the measurement target blood sample to liquid chromatography is applied to the third correlation equation instead of the second correlation equation, to correct the hemoglobin F peak value.

5. The method of measuring hemoglobin F according to claim 3,
wherein:
a third correlation equation that is a correlation equation between a hemoglobin F peak value and a sum of the hemoglobin F peak value and the estimated modified hemoglobin F peak value in the second blood sample group is preliminarily determined instead of the second correlation equation; and
a hemoglobin F peak value of a chromatogram obtained by subjecting the measurement target blood sample to liquid chromatography is applied to the third correlation equation instead of the second correlation equation, to correct the hemoglobin F peak value.

6. The measurement method according to claim 1, wherein the liquid chromatography is cation-exchange chromatography.

7. The measurement method according to claim 2, wherein the liquid chromatography is cation-exchange chromatography.

8. The measurement method according to claim 3, wherein the liquid chromatography is cation-exchange chromatography.
